(19) United States
(12) Patent Application Publication
(10) Pub. No.: US 2004/0185109 A1
(43) Pub. Date: Sep. 23, 2004

(54) Method for incorporating cationic molecules into a substrate for increasing dispersibility of cationic molecules

(76) Inventors: Charles A. Cody, Nashville, IN (US); Paul Carey, Montclair, NJ (US); Youssef Awad, North Brunswick, NJ (US)

Correspondence Address: BAKER & BOTTS, 30 ROCKEFELLER PLAZA, NEW YORK, NY, 10112

(21) Appl. No.: 10/622,327

(22) Filed: Jul. 18, 2003

Related U.S. Application Data
(60) Provisional application No. 60/397,437, filed on Jul. 18, 2002.

(51) Int. Cl. A61K 9/14; A61K 7/00

(52) U.S. Cl. 424/489; 424/401

(57) ABSTRACT

The present invention generally provides a method for increasing the dispersibility of a cationic molecule of interest through the ion exchange of the cationic molecule onto the surface of a substrate having a high surface area. The present invention further provides for the resulting compositions whereby a cationic molecule of interest has been incorporated onto the surface of a high surface area substrate and where the resulting cation/substrate (such as a cation/organoclay) composition experiences greater dispersibility in a target application system than the cationic molecule of interest alone experiences in that same application system. The method of the present invention further serves to substantially reduce the water solubility of the cationic molecule of interest by incorporating it into a high surface area substrate such as an organoclay. Also, the method of the present invention serves to improve the efficacy of the cationic molecule of interest.

### FIELD OF THE INVENTION

The present invention generally relates to a method for increasing the dispersibility of a cationic molecule of interest through the ion exchange of the cationic molecule onto the surface of a substrate having a high surface area, such as a clay. More particularly, the present invention provides a method for evenly and completely incorporating a cationic molecule of interest into a composition having an extremely large surface area, so that once the cationic molecules are incorporated, they remain non-soluble in aqueous environments. The present invention further relates to compositions resulting from the ion exchange of a cationic molecule of interest onto the surface of a clay or other high surface area substrate, wherein the remaining ion exchange capacity of the substrate is neutralized with a cationic quaternary ammonium compound, and wherein the resulting composition has a significantly enhanced dispersibility.

### BACKGROUND OF THE INVENTION

Organoclays, including chemically modified smectite-type clays such as bentonite or hectorite, are analogous to very thin sheets of paper in that the clay particles are long in width and length and have a very high surface area per unit weight. Smectite-type clays and methods for their preparation are disclosed in U.S. Pat. No. 4,664,820 to Magauran et al., which is hereby incorporated by reference in its entirety. Organoclays are further characterized in that they contain mobile organic cations at their surface, which can be readily ion-exchanged with other cations when such organoclays are placed in water. The mobile cations located on the surface of an organoclay may include, but are not limited to, Na⁺, Li⁺, K⁺, NH₄⁺, H⁺, Ca²⁺, Mg²⁺, and Fe²⁺. Since the above-listed cations are mobile, they may be replaced by other cations, such as quaternary ammonium compounds, referred to herein as “quats”, which comprise a positively charged nitrogen-containing organic ionic portion associated with a negative ion, such as Cl⁻or Br⁻.

Typically, quats ionize in water. For example, a quat such as (CH₃)₂—N⁺—[(CH₂)₁₇—CH₃]₂—Cl⁻ is able to ionize in water and exchange onto the surface of a high surface area substrate such as a clay so that the resulting organoclay has a surface that is coated with cationic organics. The surface coverage of the quat on the clay surface is so complete that organic systems will then disperse the organoclay because of the organic surface modification of the organoclay. Thus, the inclusion of a cationic organic compound such as a quat on the surface of the clay provides a surface with a high compatibility for dispersion in organic systems. In addition, the quat completes the neutralization of the clay's negative charges.

Cationic organic dyes, such as methylene blue, consist of a positive portion, the colored part, and an off-setting negative portion, an anion. When such a cationic organic dye is placed in water, it typically dissolves or dissociates into anions and cations, and the cationic portion colors the system. However, when such organic cationic dyes are used in systems other than water (for example, in organic systems), it is difficult to disperse the cationic dye because of its ionic character. The ionic character of the cationic dye, then, typically makes the dye non-soluble and non-dispersible in an organic system, and if the cationic dye is dispersed at all, it typically bleeds or is easily washed out with water.

Thus, a need exists for a method whereby an organic cationic molecule (such as a cationic organic dye) can experience greater dispersibility in a non-ionic system such as an organic system. The invention disclosed herein addresses this need.

The use of an organophilic clay gellant has been disclosed in U.S. Pat. No. 4,412,018 to Finlayson et al. In Finlayson et al., the objective of the invention described therein is to use the clay gellant as a thickening agent, and the cationic molecule portions used by Finlayson et al. are simply present to enhance the properties of the organoclay. The disclosure of Finlayson et al. does not disclose the use of an organoclay to enhance the properties of a cationic molecule of interest.

Similarly, U.S. Pat. Nos. 5,804,613 to Beall et al. and U.S. Pat. No. 6,242,500 to Lan et al. disclose an intercalate, wherein material may be added to a clay to enhance the dispersibility of the clay. Beall et al. and Lan et al. do not contemplate the enhancement of the chemical properties of a cationic molecule of interest through increasing the dispersibility of such cationic molecules.

In addition, U.S. Pat. Nos. 4,434,075 and 4,517,112 to Mardis et al. disclose modified organophilic clays but do not contemplate a method by which the dispersibility and the chemical and physical properties of a cationic molecule of interest are greatly increased and/or enhanced through the ion exchange of the cationic molecule onto the surface of an organoclay.

Thus, in all of the above-cited documents, there is no recognition or even a suggestion that the surface of an organoclay may be employed to enhance certain chemical or physical features or properties of a cationic molecule of interest. Therefore, a need exists for a method of reacting a cationic molecule of interest, such as a cationic dye, having a desired chemical property, such as coloring a system, with a high surface area substrate, such as a clay or an organoclay, via ion exchange of the cationic molecule of interest onto the surface of the clay, so that the dispersibility of the cationic molecule of interest is greatly increased and so that the ability of the cationic molecule to impart its desired chemical property to a given system is greatly enhanced. The methods and resulting compositions disclosed in the present invention address this need as well as other needs.

### SUMMARY OF THE INVENTION

The present invention relates to a method for increasing the dispersibility and enhancing the chemical and/or physical properties of a cationic molecule of interest by reacting the positively charged or cationic portion of that molecule onto the surface of a composition via ion exchange. The invention provides methods and compositions that permit a cationic molecule of interest to be evenly and completely dispersed over an extremely large surface area, and to be non-soluble in the system or application of interest. Thus, the present method provides the desired result of substantially diminishing the water solubility of the cationic molecule of interest while simultaneously keeping the cation fixed and viable over an extremely large surface.

The present invention generally provides a method (and the resulting compositions) whereby a cationic molecule of interest experiences greater dispersibility in systems where it typically is unable to disperse. For example, methylene blue, a cationic dye, typically is completely unable to color an organic system such as mineral oil. However, once methylene blue is incorporated onto the surface of an organoclay (such as Bentonite clay treated with a quaternary ammonium compound), the methylene blue/organoclay composition is now able to intensely color the mineral oil, even when methylene blue has been added at an amount as low as about 2 or 3% by weight.

Thus, it is contemplated that in some embodiments, the method of the present invention essentially converts a cationic dye into a cationic pigment so that coloring of a system can be maintained while the possibility of the dye bleeding is eliminated.

Various embodiments described herein employ a clay as the substrate having high surface area that is able to increase the dispersibility of the cationic molecule of interest. As the dispersibility of a cationic molecule of interest is increased, the ability of that cationic molecule of interest to impart a desired chemical effect to a given system is greatly enhanced. Thus, for example, if an organic cationic dye is dispersed onto the surface of a clay according to the method of the present invention, the increased dispersibility of the cationic dye results in an increase in the ability of the cationic dye to color the desired system, and both color strength and intensity will increase proportionately. Therefore, the present invention further provides an improved method of coloring a system whereby a cationic dye is complexed with a clay or an organoclay, and the resulting cation/clay or cation/organoclay composition's ability to color a given system (and to remain non-soluble in that system) is greater than that of the cationic dye alone.

In the process of the present invention, the cationic portion of the cationic molecule of interest (for example, the cationic portion of a cationic dye) is ionically exchanged and bound to the surface of the high surface area substrate, such as a clay. The remaining ion exchange capacity of the clay may be neutralized with a quat (a quaternary ammonium compound). The ion exchange of the cationic molecule onto the clay produces a high surface area cationic composition that may now be easily dispersed into an organic system.

The method of the present invention is useful for any cationic molecule that is able to exchange onto the surface of the high surface area substrate (such as a clay) and after such exchange, will not remain mobile or become ionized when in water. In certain embodiments, the cationic molecule of interest may be a positive species that completely satisfies the total cation exchange capacity of the clay, and thus no quat is needed to complete the neutralization of the negative charges located on the clay surface. When no quat is used, such resulting cation/clay compositions may have their greatest dispersibility in the form of a dry powder to be used in dry organic systems.

However, in many systems, the use of a quat in conjunction with the cationic molecule of interest leads to a greater enhancement of the dispersibility of the resulting cation/organoclay composition, especially when the target system or application system comprises organic fluids. Specifically, many cationic molecules of interest are not chemically compatible with certain application systems, such as systems comprising organic fluids. Thus, in such embodiments, a combination of quat and the cationic molecule of interest is employed. In these embodiments, the specific quat used is selected to provide compatibility between the cationic molecule of interest and the application system, and thus the quat aids in the dispersion of the organoclay/cation composition into that application system.

Embodiments such as those described above illustrate that an important objective of the method of the present invention is to find an appropriate balance between the amount of the cationic molecule of interest that is used and the amount of quat used, so that the resulting cation/organoclay composition experiences the highest level of dispersibility in systems such as organic systems, while also maximizing the loading of the cationic molecule of interest.

For example, when selecting the cationic molecule of interest, it is typically suggested to select a cationic molecule, wherein the cation/high surface area substrate composition has a solubility product constant or Kₛₚ of 10⁻² or less grams²/100 mL of H₂O, wherein the Kₛₚ is defined as [organoclay][cation of interest], and where [] denotes concentration in grams per 100 mL of water. Thus, as long as the cation/organoclay composition has the requisite Kₛₚ value, that cationic molecule of interest will be useful in the process of the present invention and can be converted into an insoluble form while simultaneously having its dispersibility and available surface area greatly increased. In certain embodiments, the determination of the increase of the available surface area of the cationic molecule of interest is made by comparing the available surface area of a dry composition containing the cationic molecule of interest with the available surface area of a cation/organoclay composition according to the present invention, which contains the cationic molecule of interest.

Cationic dyes may be the clearest examples of cationic molecules of interest that benefit from the method of the present invention. As used herein, a cationic dye refers to any cationic substance, natural or synthetic, which is soluble and is used to color various materials. For example, cationic dyes useful in the present invention include methylene blue, Basic Yellow 57, Basic Green 4, Basic Red 104, methyl green, and the like. However, the cationic molecule of interest does not have to be colored or contain a chromophore, but instead may be any positively charged portion of a molecule. An essential feature of a cationic molecule of interest that will benefit from the method of the present invention is that the positive portion of the molecule is what carries or supplies the chemical effect to be imparted to a given system.

When a cationic dye is employed as the cationic molecule of interest in the present invention, the positively charged portion of the dye is what supplies or carries the coloring effect to the system. Similarly, certain pigments, pharmaceutical compounds, catalysts, initiators, redox agents, and the like are useful in the present invention if the cationic portion of such compounds is the portion that supplies the desired chemical effect. As used herein, the term pigment describes a finely divided, water-insoluble colored substance, which is used to impart its color to the substance to which it is added. Other examples of cationic molecules of interest that are useful in the present invention include, but are not limited to, petunidin, which is an oxygen-based cationic molecule and is violet colored with a coppery luster, and tolonium chloride, which is a sulfur-based cationic molecule and has been examined both for treating bleeding disorders in clinical studies and for parathyroid identification during tyroidectomy procedures.

In embodiments where cationic medicinal agents or cationic pharmaceuticals are selected as the cationic molecule of interest, the advantages gained through the present invention are numerous. For example, for a cationic medicinal agent made up of particles where typically only the outer surface of the particles interacts with the system to be treated and where the bulk of the interior of the particles is inert, the present invention provides a much larger active fraction of the medicinal agent particles to the system at the exact same weight loading. This increase in the active fraction of the particles results from the medicinal agent being incorporated onto the surface of a high surface area substrate such as an organoclay.

The high surface area substrates, onto which the cationic molecules of interest are ion exchanged and thereby experience increased dispersibility, include clays, such as smectite-type clays, silicates, such as zeolites, and the like. Essentially, the substrate must have a high surface area and must be able to undergo cationic exchange on the surface. For example, materials such as attapulgite, vermiculate, and organic resins capable of exchanging cations may be appropriate for use in the present invention. Also, the organically modified or cationically modified substrate must be dispersible in the application system into which the cationic molecule of interest is going to be dispersed.

In certain preferred embodiments of the present invention, a smectite-type clay such as bentonite clay is employed as the substrate. The use of clay as the substrate in such embodiments provides a high surface area composition and a reactive surface, which leads to the organically modified clay being an organically dispersible substrate. As noted above, once the clay binds the cationic portion of the cationic molecule of interest to its surface (via cation exchange), the cationic molecule's water solubility is substantially diminished, and thus the dispersibility of the cationic molecule of interest into organic systems is greatly increased.

When an organoclay (or a clay that has been organically modified by a cationic organic compound such as a quat) is employed in the method of the present invention, the cation/organoclay composition comprising, for example, cationic dyes or pigments bound to the organoclay, may be used to color powders, cosmetics, toners, rubbing compounds, buffing compounds, inks, resins, coatings, paints, and the like. In addition, such organoclay-bound dyes or pigments may be used to color plastics, elastomers, extruded solids, and the like. When a cationic pharmaceutical compound is bound to an organoclay, the resulting composition may be used to treat subjects.

The method of the present invention may be carried out in several different ways. Specifically, three distinct procedures are available for incorporating the cationic molecule of interest onto the surface of a high surface area substrate such as a clay or an organoclay: (1) the cationic molecule of interest may be first mixed with a quat in water and subsequently reacted with clay to form the resulting cation/organoclay composition that is dispersible in an application system of choice; (2) a quat may be first reacted with clay in water, forming an organoclay, and the cationic molecule of interest is subsequently ion exchanged onto the organoclay surface to form the resulting cation/organoclay composition that is dispersible in an application system of choice; and (3) the cationic molecule of interest may be first ion exchanged onto the surface of the clay in water, followed by the reaction of a quat with the cation/clay complex to form the resulting cation/organoclay composition that is dispersible in a chosen application system. A fourth method involves mixing the cationic molecule of interest, the quat, the clay, and water, and then extruding the mixture through an extruder to form the reacted product. Such a process is commonly known as pugmilling.

The total amount of cation used in preferred embodiments (where this “total amount” includes the cationic molecule of interest as well as the quat) should correspond to an amount which satisfies from about 90% to about 120% of the substrate's Cation Exchange Capacity (“CEC”). CEC is described more particularly in the Detailed Description section below. When the total amount of cation used exceeds 100% of the substrate's CEC, the excess cations are adsorbed nonionically onto the surface of the substrate. The CEC values of various substrates (such as clays) used in the present invention may be measured using the well-known methylene blue test, which is also described in more detail below. Then, the CEC value for the substrate will aid in determining the total amount of cation to be used.

The present invention is further described below with respect to certain specific embodiments.

### BRIEF DESCRIPTION OF THE DRAWINGS

Further objects and advantages of the present invention will be more fully appreciated from a reading of the detailed description when considered with the accompanying drawings, wherein:

FIGS. 1(A), (B), and (C) diagrammatically show three of the methods by which a cationic molecule of interest may be incorporated onto the surface of a high surface area substrate, where the substrate in FIGS. 1(A), (B), and (C) is a clay.

### DETAILED DESCRIPTION OF THE INVENTION

The present invention relates to a method for increasing the dispersibility of and enhancing the chemical and/or physical properties of a cationic molecule of interest by ion exchanging the cationic molecule onto the surface of a substrate such as a clay surface. Specifically, the present method involves ionically binding the cationic portion of the cationic molecule of interest to the surface of a substrate, such as a clay, wherein the surface of the clay may comprise organic cationic compounds, such as quats, which serve to further neutralize the slightly negative charges located on the surface of the clay. Thus, the present invention provides a method that allows cationic molecules of interest to be completely and evenly dispersed over an extremely large surface area in a non-soluble form. The present invention further provides compositions resulting from the reaction of a cationic molecule of interest onto the surface of a highly dispersible substrate such as a clay or an organoclay (a clay that has been modified to comprise a quat).

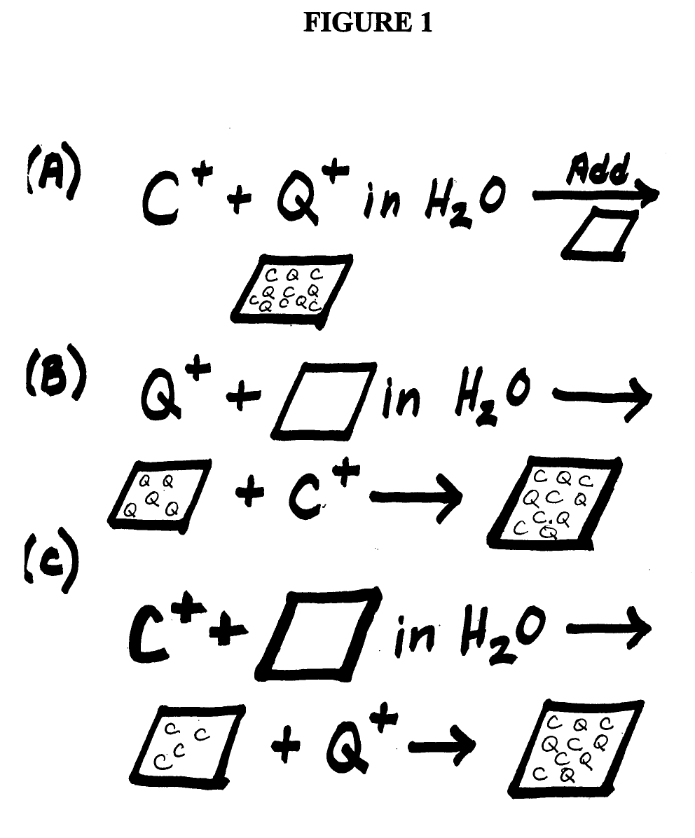

In certain embodiments of the present invention, the cationic molecule of interest is first reacted with a quat in an aqueous environment such as pure water. Subsequently, the mixture of quat and the cation of interest is added to the high surface area substrate, such as clay, to form the cation/organoclay composition that is dispersible in various application systems. These embodiments generally correspond to the reaction diagram included as FIG. 1(A).

In other embodiments, a quat is first reacted onto the surface of a high surface area substrate, such as clay, thereby forming an organoclay. This reaction takes place in water. Subsequently, the cationic molecule of interest is ion exchanged onto the surface of the organoclay to form the cation/organoclay composition that experiences enhanced dispersibility in application systems when compared to the dispersibility of the cation alone in that application system. These embodiments generally correspond to the reaction diagram included as FIG. 1(B).

In still other embodiments, the cationic molecule of interest is first ion exchanged onto the surface of a high surface area substrate, such as a clay, and this reaction takes place in water. Subsequently, a quat is ion exchanged onto the surface of the clay to form the cation/organoclay composition that is dispersible in various application systems. Such embodiments generally correspond to the reaction diagram included as FIG. 1(C). Note that in FIGS. 1(A), (B), and (C), the symbol “C⁺” stands for the cationic molecule of interest, the symbol “Q⁺” represents the quat, and the parallelogram-shaped object, which looks like a flat sheet or plate, represents clay, where clay is being used as the high surface area substrate.

In certain preferred embodiments of the present invention, a cationic dye such as methylene blue is employed as the cationic molecule of interest and a clay is used as the high surface area substrate. In such embodiments, the ion exchange of the methylene blue onto the surface of the clay is accomplished due to the cationic replacement and exchange of the mobile cations located on the clay, which are associated with the negatively charged surface of a clay platelet. Once the cationic molecule of interest (such as methylene blue) is ion exchanged onto the clay surface, the cationic molecule of interest experiences greatly enhanced dispersibility in systems such as organic systems.

As discussed earlier, many possible end uses exist for compositions formed according to the present invention. Specifically, clays and organoclays employing a dye or a pigment as the cationic molecule of interest may be used to color powders and may be used in cosmetics, toners, rubbing and buffing compounds. In addition, the cation/organoclay compositions formed according to the present invention may be used in drug applications, such as being mixed in with aspirin, Mg(OH)₂, CaCO₃, or the like. Also, the resulting cation/organoclay compositions formed herein may be dispersed, with at least low to medium intensity mixing, into paints, coatings, lubricants, resins (including polyester), alkyds, oils, greases, and various other organic fluids.

Furthermore, the dry powder form of the organoclay/cation compositions formed herein may be blended with powders, polymers, resins, and the like, and thereby used in dry form. Also, such blended powders incorporating the dry powder form of the cation/organoclay compositions formed herein may be melted or melt extruded for use in materials such as thermoplastics. For example, a blended powder incorporating a dry powder form of an organoclay/cation composition formed according to the present invention could be used to color nanocomposite-containing materials, such as the nanocomposite thermoplastic olefin materials described in Rose, J., “Nanocomposite TPO Part Is Ready to Hit the Road for GM,” Modern Plastics, (October 2001), p. 37, which is hereby incorporated by reference herein in its entirety. Specifically, in such embodiments, the colored nanocomposite thermoplastic olefin materials could be used in automotive parts as well as other applications.

Any organic cationic molecule capable of ion exchange onto the surface of the high surface area substrate (such as a clay) may be used in the method of the present invention. The positive charge on the organic cation may be +1, +2, +3, or greater, and this charge may be located on any atom within the molecular structure of the molecule such as carbon atoms, nitrogen atoms, sulfur atoms, oxygen atoms, phosphorus atoms, and the like. Representative examples of molecules comprising such a cationic portion may include but are not limited to certain catalysts, pharmaceutical compounds, reaction intermediates, dyes, pigments, initiators, and redox agents. Other examples of cationic molecules of interest that may be used in the present invention include the following: pyocyanine, a nitrogen-based cationic molecule that supplies a dark blue color in solution; phenosafranin, a nitrogen-based cationic molecule that is useful as a biological stain; active methionine, a sulfur-based cationic molecule that is medically useful as an essential nutrient and a lipotropic agent and that is useful to regulate urine pH in dogs and to treat liver disease in certain animals; gallamine triethiodide, a cationic molecule comprising 3 positively charged nitrogen atoms that is useful as a skeletal muscle relaxant for humans and other animals; dodecarbonium chloride, a nitrogen-based cationic molecule that is medically useful as an antiseptic or disinfectant; dodecyltripheynylphosphonium bromide, a phosphorous-based cationic molecule that is medically useful as a topical antifungal agent; dipropamine, a nitrogen-based cationic molecule that is medically useful for its curare-like activity; dibutoline sulfate, a nitrogen-based cationic molecule that is medically useful as an anticholinergic and for biliary spasms; cetalkonium chloride, a nitrogen-based cationic molecule that is useful as a cationic surfactant germicide or fungicide or medically as an antibacterial agent; cethexonium bromide, a nitrogen-based cationic molecule that is medically useful as an antiseptic; cetiprin, a nitrogen-based cationic molecule that is medically useful as an antispasmotic agent; cephalosporin C, a nitrogen-based cationic molecule that is medically useful for its antimicrobial properties; and celestine blue, an oxygen-based cationic molecule that is useful as a dye to dye fabrics navy-blue or as a nuclear and connective tissue stain.

In certain preferred embodiments of the present invention, a cationic dye is chosen as the cationic molecule of interest to be ion exchanged onto the surface of a substrate such as a clay. Examples of cationic dyes useful in the present invention include, but are not limited to, methylene blue, Basic Yellow 57, Basic Green 4, Basic Red 104, methyl green, and the like. In the context of the present invention, dyes that are useful herein may be represented by the general formula “AB,” wherein AB is ionically neutral, A⁺ is the cationic portion of the dye that supplies the coloration ability of that dye, and B⁻ is the balancing anion that is chosen so that the dye will be soluble in water. Thus, dyes typically undergo the following reaction in aqueous environments:

AB+H₂O→A⁺+B⁻

In addition to dyes, cationic pigments also may be ion exchanged onto the surface of a high surface area substrate, such as a clay. Pigments are typically small colored particles, possibly 0.05 μm to about 5 μm in size, which are able to color a system. Pigments typically do not experience the same problems as cationic dyes such as the problem of bleeding when in an aqueous environment. However, pigments suffer from the fact that the bulk of the pigment powder is unable to provide color to the target system because the majority of the pigment particle is buried in the interior. Thus, the method of the present invention, wherein the dispersibility of a cationic pigment is greatly increased, better allows such a pigment to color a system by increasing the surface area of the pigment available to the system.

In embodiments of the present invention employing a cationic pigment as the cationic molecule of interest, the ion exchange of the pigment onto the surface of the substrate such as an organoclay typically takes place before a neutralizing anion (B⁻) is added to the colored cationic portion of the pigment. This is because in contrast to cationic dyes, cationic pigments are typically charge-neutral and are not water soluble.

If the representative formula AB is used to describe a pigment, A⁺ is the cationic portion of the pigment which supplies the coloration, and B⁻ is a balancing or neutralizing anion that makes AB insoluble in water. Thus, when cationic pigments are to be used in the present invention, only the “A⁺” or cationic, colored part of the pigment is needed to react onto the surface of the organoclay, via cation exchange. Thus, the negative charge located on the surface of the clay actually serves as the “neutralizing anion” for the cationic portion of the pigment.

High surface area substrates that may be utilized in the practice of the present invention include, but are not limited to, clays and organoclays as well as silicates, such as zeolites. As discussed earlier, an organoclay comprises an organic cationic compound dispersed on the surface of the clay. Such organic cationic compounds useful in embodiments where an organoclay is employed as the high surface area substrate may be selected from a wide range of compounds having a positive charge localized on a single atom or a group of atoms within the compound. In certain preferred embodiments, a quaternary ammonium salt is the organic cationic compound used to modify a clay into an organoclay.

In certain embodiments, smectite-type clays, particularly bentonite clay, may be selected as the high surface area substrate. Bentonite clay is highly dispersible in water and results in numerous particles with an extremely high surface area. On average, one can approximate a bentonite clay particle in water as having the dimensions of 0.1 μm in length, 0.1 μm in width, and 10 Å in thickness. This type of clay is also well known to contain exchangeable cations on its surface. When dispersed in water, the surface exchangeable cations, such as Na+, Ca²⁺ and Mg²⁺, can be exchanged with organic cations, such as quaternary ammonium salts (“quats”), to form an organoclay. The formation and use of organoclays are described in U.S. Pat. Nos. 5,759,938 issued Jun. 2, 1998 to Cody et al.; U.S. Pat. No. 5,735,943 issued Apr. 7, 1998 to Cody et al.; U.S. Pat. No. 5,725,805 issued Mar. 10, 1998 to Kemnetz et al.; U.S. Pat. No. 5,696,292 issued Dec. 9, 1997 to Cody et al.; U.S. Pat. No. 5,667,694 issued Sep. 16, 1997 to Cody et al.; U.S. Pat. No. 5,634,969 issued Jun. 3, 1997 to Cody et al.; and U.S. Pat. No. 4,664,820 issued May 12, 1987 to Magauran et al.; all of which are incorporated herein by reference in their entirety.

Also described in the above-referenced patents are additives which may be employed to assist in further increasing the dispersibility of cationic molecules of interest through incorporating such cations into, for example, organoclay materials as disclosed herein. Examples of suitable additives include, but are not limited to, polar activators, such as acetone; preactivators, such as 1,6 hexane diol; intercalates, such as organic anions; and mixtures thereof. Such additives are also described in U.S. Pat. Nos. 5,075,033 to Cody et al.; U.S. Pat. No. 4,894,182 to Cody et al.; and U.S. Pat. No. 4,742,098 to Finlayson et al.; which are all incorporated herein by reference in their entirety.

Any clay, which can undergo ion exchange with one or more organic cations to provide binding of a cationic molecule of interest, may be used in the method and compositions of the present invention. Preferable clays include smectite-type clays, which are well known in the art and are available from a variety of sources. The clays can also be converted to the sodium form if they are not already in this form. This can conveniently be done by preparing an aqueous clay slurry and passing the slurry through a bed of cation exchange resin in the sodium form. Alternatively, the clay can be mixed with water and a soluble sodium compound, such as sodium carbonate, sodium hydroxide, or the like, and the mixture may be sheared, for example, using a pugmill or extruder. Conversion of the clay to the sodium form can be undertaken at any point before the ion-exchange with the organic cationic molecule of interest.

Smectite-type clays prepared synthetically by either a pneumatolytic or, preferably, a hydrothermal synthesis process can also be used to prepare the cationic compositions taught in the method of the present invention. Representative smectite-type clays which are useful in the present invention include, but are not limited to, the following:

Montmorillonite, having the general formula:

[(Al₄₋ₓMgₓ)Si₈O₂₀(OH)_{4-f}F_{f}]ₓR⁺

where 0.55≦x≦1.10, f≦4 and where R is selected from the group consisting of Na, Li, NH₄, and mixtures thereof;

Bentonite, having the general formula:

[(Al₄₋ₓMgₓ)(Si_{8-y}Al_{y})O₂₀(OH)_{4-f}F_{f}]_{(x+y)}R⁺

where 0<x<1.10, 0<y<1.10, 0.55≦(x+y)≦1.10, f≦4 and where R is selected from the group consisting of Na, Li, NH₄ and mixtures thereof,

Beidellite having the general formula:

[(Al_{4+y})(Si_{8-x-y}Al_{x+y})O₂₀(OH)_{4-f}F_{f}]ₓR⁺

where 0.55≦x≦1.10, 0≦y≦0.44, f≦4 and where R is selected from the group consisting of Na, Li, NH₄ and mixtures thereof;

Hectorite having the general formula:

[(Mg₆₋ₓLiₓ)Si₈O₂₀(OH)_{4-f}F_{f}]ₓR⁺

where 0.57≦x≦1.15, f≦4 and where R is selected from the group consisting of Na, Li, NH₄, and mixtures thereof;

Saponite having the general formula:

[(Mg_{6-y}Al_{y})(Si_{8-x-y}Al_{x+y})O₂₀(OH)_{4-f}F_{f}]ₓR⁺

where 0.58≦x≦1.18, 0≦y≦0.66, f≦4 and where R is selected from the group consisting of Na, Li, NH₄, and mixtures thereof; and

Stevensite having the general formula:

[(Mg₆₋ₓ)Si₈O₂₀(OH)_{4-f}F_{f}]₂ₓR⁺

where 0.28≦x≦0.57, f=4 and where R is selected from the group consisting of Na, Li, NH₄, and mixtures thereof.

The preferred clays used in the present invention are bentonite and hectorite, with bentonite being the most preferred. The clays may be synthesized hydrothermally by forming an aqueous reaction mixture in the form of a slurry containing mixed hydrous oxides or hydroxides of the desired metals with or without, as the case may be, sodium (or alternate exchangeable cations or mixtures thereof) fluoride in the proportions defined by the above formulas and the preselected values of x, y, and f for the particular synthetic smectite-type clay desired. The slurry is then placed in an autoclave and heated under autogenous pressure to a temperature within the range of approximately 100° to 325° C., preferably 275° to 300° C., for a sufficient period of time to form the desired product. Formulation times of 3 to 48 hours are typical at 300° C., depending on the particular smectite-type clay being synthesized, and the optimum time can readily be determined by pilot trials.

Representative hydrothermal processes for preparing synthetic smectite-type clays are described in U.S. Pat. Nos. 3,252,757; 3,586,478; 3,666,407; 3,671,190; 3,844,978; 3,844,979; 3,852,405; and 3,855,147; all of which are incorporated by reference in their entirety.

In embodiments where an organoclay is used as the high surface area substrate for the ion exchange of the cationic molecule of interest, a variety of organic cationic compounds may be used to modify the clay into an organoclay, which thereby enhances the dispersibility of the cationic molecule of interest. Specifically, the organic cations used to modify a clay into an organoclay in the present method must have a positive charge localized on a single atom or on a small group of atoms within the compound. The organic cation is preferably an ammonium cation which contains at least one linear or branched, saturated or unsaturated alkyl group having 12 to 22 carbon atoms. The remaining groups of the organic cationic compound are chosen from (a) linear or branched alkyl groups having 1 to 22 carbon atoms; (b) aralkyl groups which are benzyl and substituted benzyl moieties including fused ring moieties having linear or branched 1 to 22 carbon atoms in the alkyl portion of the structure; (c) aryl groups such as phenyl and substituted phenyl including fused ring aromatic substituents; (d) beta, gamma-unsaturated groups having six or less carbon atoms or hydroxyalkyl groups having two to six carbon atoms; and (e) hydrogen.

The long chain alkyl radicals may be derived from naturally occurring oils including various vegetable oils, such as corn oil, coconut oil, soybean oil, cottonseed oil, castor oil and the like, as well as various animal oils or fats such as tallow oil. The alkyl radicals may likewise be petrochemically derived, for example, from alpha olefins.

Representative examples of useful branched, saturated radicals include 12-methylstearyl and 12-ethylstearyl. Representative examples of useful branched, unsaturated radicals include 12-methyloleyl and 12-ethyloleyl. Representative examples of unbranched saturated radicals include lauryl, stearyl, tridecyl, myristyl (tetradecyl), pentadecyl, hexadecyl, hydrogenated tallow, and docosanyl. Representative examples of unbranched, unsaturated and unsubstituted radicals include oleyl, linoleyl, linolenyl, soya, and tallow.

Additional examples of aralkyl groups (or groups comprising benzyl and substituted benzyl moieties) include those materials derived from, e.g., benzyl halides; benzhydryl halides; trityl halides; α-halo-α-phenylalkanes, wherein the alkyl chain has from 1 to 22 carbon atoms, such as 1-halo-1-phenylethane, 1-halo-1-phenyl propane, and 1-halo-1-phenyloctadecane; substituted benzyl moieties, such as those moieties derived from ortho-, meta- and para-chlorobenzyl halides; para-methoxybenzyl halides; ortho-, meta- and para-nitrilobenzyl halides; ortho-, meta- and para-alkylbenzyl halides, wherein the alkyl chain contains from 1 to 22 carbon atoms; and fused ring benzyl-type moieties, such as those moieties derived from 2-halomethylnaphthalene, 9-halomethylanthracene and 9-halomethylphenanthrene, wherein the halo group would be defined as chloro, bromo, iodo, or any other such group which serves as a leaving group in the nucleophilic attack of the benzyl type moiety such that the nucleophile replaces the leaving group on the benzyl type moiety.

Examples of aryl groups would include phenyl, such as in N-alkyl and N,N-dialkyl anilines, wherein the alkyl groups contain between 1 and 22 carbon atoms; ortho-, meta- and para-nitrophenyl; ortho-, meta- and para-alkyl phenyl, wherein the alkyl group contains between 1 and 22 carbon atoms; 2-, 3-, and 4-halophenyl, wherein the halo group is defined as chloro, bromo, or iodo; 2-, 3-, and 4-carboxyphenyl and esters thereof, where the alcohol of the ester is derived from an alkyl alcohol, wherein the alkyl group contains between 1 and 22 carbon atoms; aryl such as a phenol; aralkyl such as benzyl alcohols; and fused ring aryl moieties such as naphthalene, anthracene, and phenanthrene.

The β, γ-unsaturated alkyl group may be selected from a wide range of materials. These compounds may be cyclic or acyclic, unsubstituted or substituted with aliphatic radicals containing up to 3 carbon atoms such that the total number of aliphatic carbons in the β, γ-unsaturated radical is 6 or less. The β, γ-unsaturated alkyl radical may be substituted with an aromatic ring that likewise is conjugated with the unsaturation of the β, γ-moiety, or the β, γ-radical may be substituted with both aliphatic radicals and aromatic rings.

Representative examples of cyclic β, γ-unsaturated alkyl groups include: 2-cyclohexenyl and 2-cyclopentenyl. Representative examples of acyclic β, γ-unsaturated alkyl groups containing 6 or less carbon atoms include: propargyl; allyl(2-propenyl); crotyl(2-butenyl); 2-pentenyl; 2-hexenyl; 3-methyl-2-butenyl; 3-methyl-2-pentenyl; 2,3-dimethyl-2-butenyl; 1,1-dimethyl-2-propenyl; 1,2-dimethyl propenyl; 2,4-pentadienyl; and 2,4-hexadienyl. Representative examples of acyclic-aromatic substituted compounds include: cinnamyl(3-phenyl-2-propenyl); 2-phenyl-2-propenyl; and 3-(4-methoxyphenyl)-2-propenyl. Representative examples of aromatic and aliphatic substituted materials include: 3-phenyl-2-cyclohexenyl; 3-phenyl-2-cyclopentenyl; 1,1-dimethyl-3-phenyl-2-propenyl; 1,1,2-trimethyl-3-phenyl-2-propenyl; 2,3-dimethyl-3-phenyl-2-propenyl; 3,3-dimethyl-2-phenyl-2-propenyl; and 3-phenyl-2-butenyl.

The hydroxyalkyl group may be selected from a hydroxyl substituted aliphatic radical, wherein the hydroxyl is not substituted at the carbon adjacent to the positively charged atom, and the group has from 2 to 6 aliphatic carbons. The alkyl group may be substituted with an aromatic ring independently from the 2 to 6 aliphatic carbons. Representative examples include: 2-hydroxyethyl (ethanol); 3-hydroxypropyl; 4-hydroxypentyl; 6-hydroxyhexyl; 2-hydroxypropyl (isopropanol); 2-hydroxybutyl; 2-hydroxypentyl; 2-hydroxyhexyl; 2-hydroxycyclohexyl; 3-hydroxycyclohexyl; 4-hydroxycyclohexyl; 2-hydroxycyclopentyl; 3-hydroxycyclopentyl; 2-methyl-2-hydroxypropyl; 1,1,2-trimethyl-2-hydroxypropyl; 2-phenyl-2-hydroxyethyl; 3-methyl-2-hydroxybutyl; and 5-hydroxy-2-pentenyl.

The organic cation used when modifying a clay into an organoclay for use the present invention may thus be considered as having at least one of the following formulae:

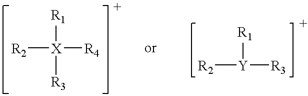

wherein X is nitrogen or phosphorus, Y is sulfur, R₁ is the long chain alkyl group and R₂, R₃ and R₄ are representative of the other possible groups described above.

A preferred organic cation employed for modifying a clay substrate into an organoclay may contain at least one linear or branched, saturated or unsaturated alkyl group having 12 to 22 carbon atoms and at least one linear or branched, saturated or unsaturated alkyl group having 1 to 12 carbon atoms. The preferred organic cationic compound may also contain at least one aralkyl group having a linear or branched, saturated or unsaturated alkyl group having 1 to 12 carbons in the alkyl portion. Mixtures of these cations may also be used.

Especially preferred organic cationic compounds include ammonium cationic compounds, wherein R₁ and R₂ are hydrogenated tallow and R₃ and R₄ are methyl, or wherein R₁ is hydrogenated tallow, R₂ is benzyl and R₃ and R₄ are methyl or a mixture thereof such as 90% (equivalents) of the former and 10% (equivalents) of the latter.

Specifically, in embodiments of the present invention where a quat is employed to modify a clay into an organoclay, a quat such as dimethyl dihydrogenated tallow quat may used for dispersion into non-polar organics. Furthermore, a dimethyl tallow benzyl quat may be used for dispersion into aromatic systems. Thus, the specific quat to be employed is selected with respect to the nature of the system into which the cation/organoclay composition will be dispersed.

As earlier mentioned, the organic cationic compound, such as a quat, is associated with an anionic portion, which portion will not adversely affect the reaction product or the recovery of the same. Such anions may include chloride, bromide, iodide, hydroxyl, nitrite and acetate in amounts sufficient to neutralize the organic cation.

In embodiments where a quaternary ammonium salt is used to modify the composition such as a clay into an organoclay, the preparation of the quaternary ammonium salt can be achieved by techniques that are well-known in the art. For example, when preparing a quaternary ammonium salt, one skilled in the art would prepare a dialkyl secondary amine, for example, by the hydrogenation of nitrites (see U.S. Pat. No. 2,355,356, which is incorporated herein by reference in its entirety) and then form the methyl dialkyl tertiary amine by reductive alkylation using formaldehyde as a source of the methyl radical. According to procedures set forth in U.S. Pat. Nos. 3,136,819 and 2,775,617, which are incorporated herein by reference in their entirety, quaternary amine halide may then be formed by adding benzyl chloride or benzyl bromide to the tertiary amine.

As is well known in the art, the reaction with benzyl chloride or benzyl bromide can be completed by adding a minor amount of methylene chloride to the reaction mixture so that a blend of products that are predominantly benzyl substituted is obtained. This blend may then be used without further separation of components to prepare the organophilic clay.

Illustrative of the numerous patents which describe organic cationic salts, their manner of preparation and their use in the preparation of organophilic clays are commonly assigned U.S. Pat. Nos. 2,966,506; 4,081,496; 4,105,578; 4,116,866; 4,208,218; 4,391,637; 4,410,364; 4,412,018; 4,434,075; 4,434,076; 4,450,095; and 4,517,112; all of which are incorporated herein by reference in their entirety.

The amount of the organic cationic compound (such as a quat) to be reacted with the smectite-type clay depends upon the specific clay being employed. As seen in the Examples below, the optimal clay:quat ratio may be determined using the well-known methylene blue spot test. The end point of this spot test is used to calculate the Cation Exchange Capacity (or “CEC”) for a given type of smectite-type clay. This CEC value is thereby used in calculating the optimal clay:quat ratio for the specific clay.

The compositions of the invention include a wide range of cationic molecules of interest wherein the cationic molecule of interest carries or supplies the chemical effect to be imparted to a given system. Examples of cationic molecules of interest include, but are not limited to, pigments, pharmaceutical compounds, catalysts, initiators, Redox agents, dyes and the like. The sought after chemical effect, such as coloring for dyes and/or pigments or medicinal activity can be quantitatively measured by a number of techniques which are specific to the chemical activity sought. In general, for each specific molecule of interest, a series of quantitative measurements are carried out on the pure chemical of interest and on the inventive compositions at equal concentrations of the chemical molecule of interest, then the measurements compared to determine the relative improvement. For example, to quantitate the decrease in water solubility of the inventive composition versus, for example pure dye itself, one can employ ultra violet visible spectroscopy to measure the relative intensity or absorption of the dye itself at a given concentration in water versus that of the same dye concentration in the inventive composition in water after filtering the composition in water to remove solids.

To measure the decreased water leachability, the inventive composition and the chemical of interest itself can be dispersed into an application system of interest. Then soxhlet extraction can be carried out on both application samples and the extracted water can be analyzed for the chemical ingredient of interest by a variety of techniques such as ultraviolet visible analysis of the water extract, determining the residue weight upon drying the water extracts etc.

The method and compositions of the present invention may be better understood through the working Examples detailed below. These Examples are intended to illustrate the invention and should not be construed as limiting the invention in any way.

### EXAMPLES

### Example 1

### General Determination of Useful Cationic Compounds

At the outset of the method of the present invention, it must first be determined if a particular compound or molecule will be able to undergo the method of enhancing its dispersibility and thereby its cationic properties as disclosed herein. Thus, a determination must be made (for example, if the ionic character of a certain compound is unknown) whether or not the ion or molecule comprises an cationic portion that will ion exchange onto the surface of a substrate such as a clay or an organoclay. Thus, the molecule or ion can be tested in order to determine whether it has a cationic character (and thus can undergo the present method) or an anionic character or a neutral character. In testing certain molecules or ions, the procedure described below was employed.

Six samples were tested for whether or not they may undergo the process of the present invention, and these samples included: Jarocol Straw Yellow dye; D&C Red No. 22 dye; FD&C Blue No. 1 dye; Methylene Blue laboratory reagent dye; FD&C Yellow No. 5 dye; and Lithol Rubine B. Specifically, D&C Red No. 22 is a well-known xanthene color (CAS Number 548-36-5) having the empirical formula C₂₀H₈Br₄O₅. 2Na. FD&C Blue No. 1 is a well-known triphenylmethane color (CAS Number 3844-45-9) having the empirical formula C₃₇H₃₆N₂O₉S₃.2Na. FD&C Yellow No. 5 dye (CAS Number 1934-21 -0) is a well-known pyrazole color having the empirical formula C₁₆H₁₂N₄O₉S₂.3Na. Also, for the Lithol Rubine B, the complex that is normally used to form Lithol Rubine B was used before complexation with Ca²⁺.

Using Jarocol Straw Yellow dye as a representative example, four 1 mg samples of the dye were weighed and placed in 4 test tubes. Then, 10 mg of an organoclay powder were added to the first test tube; 10 mg of “clean” clay (or clay that does not comprise a quaternary ammonium compound) were added to the second test tube; 10 mg of quat were added to the third test tube; and the fourth test tube contained only the Jarocol Straw Yellow dye sample.

Subsequently, 10 mL of water were added to each of the four test tubes, and all of the samples were mixed well for 30 seconds. The samples from each of the test tubes were then centrifuged for 15 minutes in a laboratory scale centrifuge.

Next, the resulting centrifuged samples were examined and analyzed (by being compared to the control sample) to determine whether the Jarocol Straw Yellow dye reacted with the organoclay, the clean clay, or the quat, so that it could then be decided if Jarocol Straw Yellow dye has the requisite cationic character to ion exchange onto the surface of a clay and thereby have increased dispersibility in various application systems.

The analysis herein was performed visually, since the six samples tested were dyes. However, non-dye samples may be analyzed through infrared spectroscopy, differential scanning calorimetry (DSC), gas chromatography, UV spectroscopy, thermogravimetric analysis, or the like. For example, for a non-dye sample, the clay may be separated from the water, both parts may be taken to dryness, and the IR spectrum of each may be recorded.

Just as Jarocol Straw Yellow dye was tested using the four test tubes, the other five dye samples were similarly tested. The results are shown below in Table 1:

TABLE 1

| | Test Tube 1 | Test Tube 2 | | Test Tube 4 |
|---|---|---|---|---|
| | (10 mg | (10 mg | Test Tube 3 | (Control, |
| Sample (1 mg) | Organoclay) | “Clean” clay) | (10 mg Quat) | sample only) |
| Jarocol Straw | Organoclay was | Clay layer was | Quat had a | Water was dark |
| Yellow dye | light yellow; | yellow; water | slight tint of | yellow |
| | water layer also | layer was clear | yellow; water | |
| | light yellow | | layer was dark | |
| | | | yellow | |
| D&C Red No. | Organoclay | Clay layer had | Quat layer was | Water was dark |
| 22 dye | layer was red; | its natural | light pink; | red |
| | water layer was | color; water | water layer was | |
| | clear | layer was red | red | |
| FD&C Blue | Organoclay | Clay layer had | Quat layer was | Water was dark |
| No. 1 dye | layer was blue; | its natural | light blue; | blue |
| | water layer was | color; water | water layer was | |
| | light blue | layer was blue | blue | |
| Methylene | Organoclay | Clay layer was | Quat layer was | Water was dark |
| Blue, | layer was blue; | dark blue; | light blue; | blue |
| laboratory | water layer also | water layer was | water layer was | |
| reagent dye | blue | clear | dark blue | |
| FD&C Yellow | Organoclay | Clay layer had | Quat layer was | Water was dark |
| No. 5 dye | layer was | its natural | light yellow; | yellow |
| | yellow; water | color; water | water layer was | |
| | layer was clear | layer was | yellow | |
| | | yellow | | |
| Lithol Rubine B | Organoclay | Clay layer had | Quat layer was | Water was red |
| | layer was red; | its natural | red; water layer | |
| | water layer was | color; water | was clear | |
| | light red | layer was red | | |

The visual results recorded in Table 1 above reveal that of the six samples tested, Jarocol Straw Yellow dye and Methylene Blue laboratory reagent dye have the requisite cationic character that is needed for the method of the present invention. This is because Test Tube 2 for both of these samples showed a clay layer which was colored and a water layer that was clear, showing that these dyes react with or ion exchange with the “clean” clay since, as described above, samples of clay readily undergo cation exchange and surface modification by cationic compounds.

These visual results further showed that Lithol Rubine B formed a highly insoluble reaction product with the quat. This is evident by looking at the results for Test Tube 3 for Lithol Rubine B where the quat layer was dark red and the water layer was clear. Thus, the Lithol Rubine B had reacted with the cationic quat compound, showing that it is anionic in nature.

Furthermore, observations of all of the test tubes for the samples of D&C Red No. 22 dye, FD&C Blue No. 1 dye, and FD&C Yellow No. 5 dye showed that these compounds exhibit anionic character to some extent. Specifically, Test Tube 1 for each of these 3 dye samples shows a reaction of the organoclay and the dye. Likewise, Test Tube 2 for each of these 3 dyes reveals that these 3 dyes have no affinity for the “clean” clay, which is further evidence that the colored portion of each of these 3 dyes is anionic in nature. These 3 dyes did not result in a reaction product with quat as highly insoluble as the reaction product of Lithol Rubine B and quat. This can be seen by comparing Test Tube 3 for these 3 dyes with Test Tube 3 for Lithol Rubine B. Thus, D&C Red No. 22, FD&C Blue No. 1, and FD&C Yellow No. 5 react with quat to an extent that reveals that these 3 dyes exhibit some anionic character.

Thus, such testing described in the above Example enables one to determine what compounds or molecules have the requisite cationic character to benefit from the present invention whereby the dispersibility of such cationic molecules of interest in various application systems is significantly increased.

### Example 2

### Preparation of the Bentonite Clay Slurry

As described in detail above, the use of a clay is preferred in certain embodiments of the present invention as the high surface area substrate for use in increasing the dispersibility of a cationic molecule of interest. Thus, it is necessary to understand how such a clay is prepared. The following two methods were employed for preparing a slurry of Bentonite clay:

Method 1: Solid bentonite clay was dispersed by slowly mixing about 3% by weight of bentonite in 97% by weight of water at room temperature. This mixture was mixed for 8 hours in a high-speed mixer in order to obtain a clay slurry. (The mixture may also be sheared in a high-shear device such as a Manton Gaulin Homogenizer in order to obtain a clay slurry.) Possibly, this mixing step aids in separating the clay into individual platelets of clay.

Subsequently, the clay slurry was separated by decanting, whereby the top fraction contained the clay slurry to be collected and used, and the waste that settled to the bottom was discarded. A small portion of the collected clay slurry was then weighed and placed in an oven for 2 hours at 100° C. in order to evaporate out all the water. The dried clay was then weighed to determine the solid weight percentage of the clay in the slurry. The solid weight percentage of the clay is typically from about 1 to about 4 or 5% by weight of the clay slurry.

Method 2: In this method, the clay slurry was prepared according to Method 1 above; however, samples of the slurry were centrifuged for various time periods (ranging from 1 minute to 9 minutes) to determine the time needed to remove most of the large undissolved foreign particles, as observed under microscope. The optimum time for centrifugation was determined to be about 5 minutes, and thus the entire clay slurry sample was centrifuged for about 5 minutes. The solid weight percentage of the bentonite clay slurry was then determined as described in Method 1 above. Herein, the solid weight percentage of the clay was found to be about 1.57% by weight of the clay slurry.

### Example 3

### Determination of Optimal Clay:Quat Ratio

As discussed above, in certain embodiments of the present invention, it is preferred to use an organic cationic compound such as a quat in conjunction with the cationic molecule of interest (such as a cationic dye), so that the quat may further neutralize the remaining negative charges on the surface of the clay or other substrate. Thus, in such embodiments, it must be determined what amount of quat to use with respect to the amount of clay being used and with respect to the amount of the cationic molecule of interest being used.

Thus, in the present Example, the optimal clay:quat ratio was determined for various samples of standard Bentonite clay, sheared standard Bentonite clay, white Bentonite clay (Southern Clay Bentonite L-400) and milled white Bentonite clay. This determination employed the Methylene Blue Spot Test, wherein a standardized solution of methylene blue (which is an example of a cationic molecule of interest to be used in the present invention) was slowly added to a fixed amount of clay. The end points observed reflected experimental volumes of methylene blue added to the clay slurry which were then used to calculate the Cationic Exchange Capacity (CEC) for the given sample of clay.

In this Example, 10 grams of each clay slurry, having a known solids content of approximately 3% by weight, were weighed into a 250 mL Erlenrmeyer flask. Then, approximately 50 mL of distilled water were added, and each clay slurry sample was stirred using a magnetic stirrer. Subsequently, 2 mL of 5 N sulfuric acid were added to each sample, and the samples were stirred.

For each clay sample, a few drops of a standardized methylene blue solution (wherein 1 mL=0.01 milliequivalents or mEq) were added from a graduated burette, which began the process of allowing the methylene blue to ion exchange onto the surface of the clay. Each sample was allowed to mix and then the flask was washed down with distilled water. While the solids were suspended, one drop of the liquid from each sample was removed with a stirring rod and placed on filter paper (Whatman #1 filter paper). Each sample was labeled according to its respective burette reading in increments of 0.1 mL. At this point, no greenish-blue halo should be seen surrounding the dyed solids.

Subsequently, increments of 0.2 to 0.5 mL of the methylene blue solution were added to each sample, with stirring, at least 5 minutes after each previous addition. After each addition of the methylene blue solution, the flask was washed down with distilled water. After 5 minutes of stirring, the spot test was repeated on filter paper, and the respective burette reading for each spot test was recorded for each of the samples.

When a faint greenish-blue halo appeared surrounding the suspended solids of the spot test, the mixture was allowed to stir for an additional 10 minutes, and the spot test was repeated. When the halo persisted, this indicated that the end point had been exceeded, and the test was complete. The volume of methylene blue used was recorded.

Thus, the saturation point of the ion exchange of the methylene blue dye onto the surface of the clay was determined by adding an excess of the methylene blue dye. This amount of methylene blue solution needed to reach the end point was used to calculate the Cation Exchange Capacity (“CEC”) for each clay being studied. Specifically, the CEC for each clay was calculated as follows:

$$CEC = \frac{\begin{matrix}amount of methylene blue ( mL ) \times \\ standardized concentration ( meth . blue ) \times 100\end{matrix}}{( g of clay slurry \times \% solids )}$$

wherein the CEC is expressed as milliequivalents (or mEq) of methylene blue per 100 grams of clay. The values obtained for the CEC of each clay are illustrated in Table 2 below.

Determining the CEC for each clay sample was important because the CEC values for each clay sample in turn allowed the calculation of the optimal clay:quat ratio for each given clay. Specifically, the CEC values were used as follows to calculate values for the optimal clay:quat ratio for each clay:

$$Clay \text{:} Quat Ratio = \frac{CEC / 1 g Clay}{1000} \times \begin{matrix} 555 ( Molecular Weight \\ of Quat )\end{matrix}$$

The molecular weight of 555 represents the molecular weight of Adogen 442, the quat of choice for the calculations performed herein. The results of each determination of the optimal clay:quat ratio for the various clays are shown below in Table 2.

TABLE 2

| | | | Clay/Quat Ratio |
|---|---|---|---|
| | Particle Size | CEC | (Adogen 442 used |
| | Average (μm) | (mEq/100 g Clay) | as the quat) |
| Standard Bentonite Clay | 3.108 | 141.1 | 1:0.7825 |
| Sheared Standard Bentonite Clay | 1.624 | 160.0 | 1:0.8881 |
| White Bentonite Clay | 7.374 | 74.3 | 1:0.4124 |
| Milled White Bentonite Clay | 0.633 | 77.3 | 1:0.4290 |

The clay/quat ratio values listed in Table 2 above thus represent approximate calculated equivalent values for the quat Adogen 442, based on weight ratio.

Using the above information, the CEC for samples of standard Bentonite clay is shown to be approximately 141 mEq per 100 grams of clay. This value is important because in further experiments involving samples of standard Bentonite clay, the cationic dye such as methylene blue was utilized at an amount of about 10% of the total CEC for the Bentonite Clay. In these experiments, the remaining 90% of the total CEC of the clay was reserved for neutralization by a quat, such as quaternary ammonium hydrogenated ditallow quat.

Specifically, the molecular weight of the cationic molecule of interest allows one to calculate how much of that cationic molecule is needed in order to satisfy a desired percentage of the clay's CEC. For example, the following calculations may be performed to determine how much cationic molecule of interest is needed for a given sample of clay: As shown above, the CEC of a given type of clay may be determined by the methylene blue test. Specifically, the CEC value for Standard Bentonite Clay was found to be 141.1 mEq/100 g clay. One may decide to add methylene blue to a sample of Standard Bentonite Clay so as to satisfy 10% of that clay's overall CEC. Thus, calculations may be performed as follows:

10% (141.1)=14.11 mEq/100 clay (to be satisfied by methylene blue);

$$\frac{14.11 m Eq}{100 g clay} ( \frac{1 Eq}{1000 mEq} ) = \frac{\begin{matrix}0.01411 Eq of charge to be \\ satisfied by methylene blue\end{matrix}}{100 g clay} ;$$

$$\frac{.01411 Eq methylene blue}{100 g clay} ( \frac{\begin{matrix}320 grams ( or MW of methylene \\ blue )\end{matrix}}{\begin{matrix}1 Eq . meth . blue ( or essentially 1 \\ mole methylene blue )\end{matrix}} )$$

$$\begin{matrix}= 4.515 g methylene blue to be added per 100 g Standard \\ Bentonite Clay .\end{matrix}$$

Thus, as shown above, the determination of the CEC for a given clay aids in determining how much quat to be added to modify the clay into an organoclay as well as how much of the cationic molecule of interest should be added to satisfy a given percentage of the clay's CEC.

The results shown in Table 2 above further indicate that reducing the mean particle size value of the standard Bentonite clay particles had the effect of increasing the CEC of the clay. The shearing to create the Sheared Standard Bentonite Clay sample (wherein the shearing took place by using a Manton-Gaulin Homogenizer) served to reduce the mean particle size of the Standard Bentonite Clay from 3.108 μm to 1.624 μm, and the CEC was thereby increased approximately 12%. Thus, the reduction of particle size was important for increasing the CEC because of the increase in exposed surface area available for cationic exchange.

The effects of increased surface area were not quite as apparent for the white Bentonite clay, which exhibited only a minor increase in its CEC when milled in a Laboratory Horizontal Mill. The 90% reduction in mean particle size, resulting from the milling of the white Bentonite clay, reflected only about a 4% increase in CEC.

The above findings, therefore, aided in the present invention in that the optimal clay:quat ratios for various grades of clay were determined, and the clay:quat ratios were predicted as a function of particle size. Furthermore, the above findings provided the CEC values for various clay samples so that those CEC values may be used to determine the amount of the cationic molecule of interest to use in relation to the amount of quat to be used to further neutralize the negative charges on the surface of the clay particles.

### Example 4

### Preparation of Organoclay

As previously discussed, an organoclay (wherein the surface of a clay has been modified to comprise a quat) is used in certain preferred embodiments of the present invention as the substrate to increase the dispersibility of the cationic molecule of interest. Thus, it is necessary to understand the steps involved in the preparation of the organoclay.

In the present Example, an organoclay was prepared using samples of the clay slurry prepared in Example 2 above and using the experimental calculations and data for CEC values and optimal clay:quat ratios from Example 3 above. First, a portion of the bentonite clay slurry from Example 2, Method 2 above was weighed, heated to 55° C., and mixed in a blender at high speed. Using the solid weight percentage of the clay (1.57%, obtained from the procedure above in Example 2), 37.5% by weight of quat was added to 62.5% by weight of the clay slurry to obtain a clay:quat solid weight ratio of 1.0:0.75. As thoroughly discussed above in Example 3, the amount of quat needed to organically modify the bentonite clay was determined by the Methylene Blue Spot Test method.

After mixing for an additional 5 minutes, the mixture was allowed to sit for 30 minutes. Thereafter, the material floating at the top (the organoclay that had formed) was collected, filtered, and washed with water. The resulting dried solids were ground using a mortar and pestle to obtain a fine powder of the organoclay.

At this point, particle size analysis may be performed to determine the dispersibility of the organoclay in a chosen application system. For example, a sample of the organoclay fine powder may be dispersed into mineral oil, whereby mineral oil acts as the application system into which it may normally be difficult for the cationic molecule of interest, such as a cationic dye, to disperse. The dispersion of the organoclay powder in mineral oil acts as the “control” sample, and it is expected that dispersibility of the organoclay into the mineral oil will be relatively high because of the organic character of both the organoclay and the mineral oil. Thus, for the control sample (or the sample of organoclay dispersed in mineral oil), the particle size mean value should be low, showing that the organoclay particles readily disperse into the mineral oil and do not agglomerate.

An experimental sample may then be prepared and subsequently compared to the control sample formed above. Specifically, the cationic molecule of interest is first chosen. Herein, methylene blue is used as an example. As described earlier, the user may desire to satisfy about 10% of the clay's CEC with the cationic molecule of interest (herein, methylene blue), and thus calculations are performed to determine how much methylene blue to add to the system.

Subsequently, the experimental sample of the methylene blue/organoclay composition that has been dried may be dispersed into mineral oil. Particle size analysis is then performed on a sample of the mineral oil dispersal of the methylene blue/organoclay composition. The particle size measurements are compared to those described above for the control. Specifically, it is desirable for the particle size measurements to approximate those of the control. This will indicate to the user that the methylene blue/organoclay composition dispersed into the mineral oil just as well as the organoclay alone did. Such results will show that even though a cationic dye such as methylene blue is typically unable to disperse in a system such as mineral oil, the use of the organoclay serves to increase the dispersibility of the cationic dye into an organic system such as mineral oil and thereby increase the ability of the cationic dye to color the system. Thus, in general, the particle size mean value and the particle size distribution data and curves for the organoclay that has the cationic molecule of interest incorporated onto its surface should approximate the particle size data for the organoclay alone, thereby indicating successful dispersion of the cation/organoclay composition into the system, such as mineral oil.

To measure the particle size (and thereby the dispersibility) of both the control sample and the experimental sample, a light scattering method was employed. The light scattering methodology included the use of a computerized Malvern particle size analyzer, in which a small amount of each of the control sample and the experimental sample was analyzed. A Malvern Mastersizer 2000 dry unit Scirocco 2000 Model #APA 2000, commercially available from Malvern Instruments Ltd. in Worchestershire, United Kingdom, was used to perform the particle size analysis. Both dry and wet samples were tested according to this method.

For dry samples of either the organoclay powder or the cation/organoclay powder, the following procedure was used. (Note that this procedure describes particle size analysis of the dry organoclay powder and the dry cation/organoclay powder before any dispersal into a liquid application system, such as the mineral oil described above.) First, both the feed tray and feed chamber were cleaned. Next, from about 2 to about 4 grams of the sample of organoclay powder or cation/organoclay powder are loaded into the feed tray. After selecting the Dry SOP (Standard Operating Procedure) and entering the appropriate label or identification information, analysis of the sample of dry organoclay powder was initiated by right-clicking on the start icon. The Dry SOP parameters are provided in Table 3 below:

TABLE 3
Dry SOP

| | | |
|---|---|---|
| Criteria | Setting | Value |

| | | |
|---|---|---|
| Sample Selection | Scirocco 2000(A) | |
| Material | Cation/Quat/Clay or Quat/Clay | |
| | Refractive Index | 1.38 |
| | Absorption | 0.1 |
| Labels | Factory Settings | |
| Reports & Saving | Factory Settings | |
| Measurement | Measurement Time | 12 seconds |
| | Measurement Snaps | 12,000 |
| | Background Time | 12 seconds |
| | Background Snaps | 12,000 |
| Sampler Settings | Sample Tray | General Purpose |
| | | (<200 g) |
| | Dispersive Air Pressure | 3 Bar |
| | Aliquots | Single |
| | Vibration Feed Rate | 40% |
| | Measurement Cycle | Single |

Upon completion of the analysis, a graph representing the particle size distribution data and the corresponding volume percent data may be obtained by selecting the records tab, right-clicking to highlight the desired record, and then selecting the results analysis (BU) tab. As described earlier, the particle size distribution data for the sample of dry cation/organoclay powder can be compared to the particle size distribution data for the sample of dry organoclay powder, which acts as the control. The particle size distribution data for the dry cation/organoclay powder should be very similar to the particle size distribution data for the dry organoclay powder.

For “wet” samples, or samples wherein the dry organoclay powder or the dry cation/organoclay powder has been dispersed into a liquid, similar particle size analysis procedures may be employed. Specifically, as described above, mineral oil may be chosen as the dispersant for the “application system of interest” into which the cationic molecule of interest may typically experience difficulty in dispersing. Thus, the particle size distribution data obtained for “wet” samples shows how well the experimental sample (cation/organoclay powder) disperses in mineral oil as compared to how well the control sample (organoclay powder) disperses in mineral oil.

The Wet SOP (Standard Operating Procedure) for the Malvern Mastersizer is selected, and a manual measurement is initiated by first selecting the options icon. The Wet SOP parameters are provided below in Table 4. After entering the appropriate information (for example, what material is under analysis and what liquid dispersant is being employed), the liquid sample well is checked to ensure that it was empty. If the sample well is not empty, it may be drained by right-clicking the empty button on the accessory menu. The empty liquid sample well may then be cleaned by clicking the clean icon.

Next, the proper liquid is selected to flush the Hydro Unit. Using a pipette, the wet sample (either the experimental sample or the control sample) is slowly transferred into the sample well until the system prompts the user to stop adding more of the sample and to initiate analysis. Analysis of the wet sample was initiated by right-clicking the start icon.

TABLE 4
Wet SOP

| | | |
|---|---|---|
| Criteria | Setting | Value |

| | | |
|---|---|---|
| Sample Selection | Hydro 2000S(A) | |
| Material | Cation/Quat/Clay | |
| | or Quat/Clay | |
| | Refractive Index | 1.38 |
| | Absorption | 0.1 |
| Dispersant Name* | Mineral Oil | |
| | Refractive Index | 1.4 |
| Labels | Factory Settings | |
| Reports & Saving | Factory Settings | |
| Measurement | Measurement Time | 12 seconds |
| | Measurement Snaps | 12,000 |
| | Background Time | 12 seconds |
| | Background Snaps | 12,000 |
| Sampler Settings | Pump/Stir Speed | 2500 RPM |
| | Tip Displacement | 100% |
| | Ultrasonics | Checked pre-measurement |
| | | 20 sec. |
| | Tank Fill | Manual |
| Cycles | Aliquots | Single |
| | Measurements | 2 per aliquot |
| | Cleaning | Before each aliquot |
| | | (check enable) |
| | Clean Mode | Manual |
| | Measurement Cycle | Multiple |
| | Delay | 10 Seconds |

*The dispersant name and its refractive index can be changed for a particular dispersant used.

Upon completion of the analysis, a graph representing the particle size distribution data and the corresponding volume percent data may be obtained by selecting the records tab, right-clicking to highlight the desired record, and then selecting the results analysis (BU) tab.

As described above, the particle size distribution data for the sample of dry cation/organoclay powder dispersed in mineral oil should approximate the particle size distribution data for the control sample of dry organoclay powder dispersed in mineral oil. This will indicate that by being reacted onto the surface of the organoclay, the cationic molecule of interest has been provided with enhanced dispersibility, and thereby enhanced ability to impart its favorable chemical and/or physical properties to a given application system (such as mineral oil).

### Example 5

### Analysis of Whether Changing the Order of Ingredients Affects Cation/Organoclay Compositions

In the present Example, an analysis was performed to determine whether changing the order of the addition of the ingredients of the cation/organoclay composition affects the properties of the composition. Specifically, the present Example determined whether the coloration properties of methylene blue, a cationic dye, were changed when the methylene blue underwent ion exchange onto the surface of an organoclay, wherein the order of the addition of the various ingredients was varied.

In this Example, samples of submicron white clay slurry were used as the substrate. This clay slurry was made by mixing 1.62% by weight solid Bentolite L 400 clay in water and subsequently mixing the clay slurry in a horizontal media mill. After mixing, the mean particle size value of the clay particles was 0.78 μm, and particle size analysis showed that 69.23% of the clay particles were below 1.00 μm in size. The quat used in the present Example was dimethyl dihydrogenated tallow quaternary amine chloride (abbreviated as “2M2HT quat”), and it was used in a fixed amount of 1 part clay to 0.65 parts quat by weight.

The cationic molecule of interest used in this Example was methylene blue dye, an organic cationic dye, and the methylene blue was added in a fixed amount so as to satisfy 10% of the clay's CEC (as described in more detail above in Example 3).

The general procedures involved in this Example for all four of the samples (despite the order in which the ingredients were added) included placing a 50 gram sample of the submicron white clay slurry described above in a glass beaker and adding 300 mL of water to the slurry. A spatula was used to mix the clay slurry and the water. The mixture was placed on a hot plate in order to heat the diluted slurry up to 55° C., and a magnetic stirrer was used for stirring during heating. The hot slurry was transferred to a Waring blender and mixed for one minute at Speed 4. The quat was dissolved in 100 mL of hot water in a separate glass beaker.

Then, once the quat was dissolved, it was added to the blender either before, after, or at the same time that the methylene blue dye was added to the blender. Then, the mixture was allowed to mix for another 5 minutes. The sample was then allowed to sit for 30 minutes, after which it was filtered and washed. The filter cake was dried in an oven at 50° C. The laboratory micro mill was then used to grind each powder for one minute.

The general procedure described above was used to obtain four different samples, and during the preparation of these four samples, the order of the addition of the ingredients was varied. These four samples and the order of the addition of their respective ingredients are listed below in Table 5.

TABLE 5

| Sample | |
|---|---|
| No. | Order of Addition of Ingredients |
| 1 | Hot clay slurry, then methylene blue, then quat. |
| 2 | Hot clay slurry, then quat, then methylene blue. |
| 3 | Hot clay slurry, then quat and methylene blue were added |
| | together to the hot clay slurry. |
| 4 | Hot clay slurry, then quat, then methylene blue; |
| | same as Sample No. 2, however, an additional 10% of the |
| | amount of quat was added from the beginning. |

Thus, at this point, four different organoclay powders containing methylene blue as the cationic molecule of interest had been obtained.

Subsequently, the following eight solvents were used to disperse Samples 1-4: (1) pure water; (2) water/HCl; (3) water/NH₄; (4) isopropyl alcohol; (5) acetone; (6) toluene; (7) mineral spirit; and (8) alkyd oil. In this part of the Example, 0.1 grams of each powder obtained above (comprising the methylene blue/organoclay composition) was added to 13 mL of each solvent in a test tube. Each test tube was covered and shaken by hand for 30 seconds. The test tubes were left to stand for 24 hours in order to allow the samples to fully disperse and reach equilibrium.

At this point, each sample was then mixed again for another 30 seconds. The samples were then centrifuged for 10 minutes at Speed 90. The liquids were transferred to clean test tubes, since some particles remained affixed to the sides of the original test tubes. The coloration of each of the samples was then measured by visual observation, and the results of these observations are summarized in Table 6 below.

TABLE 6

| Sample | | Water/ | Water/ | Isopropyl | | | Mineral | Alkyd |
|---|---|---|---|---|---|---|---|---|
| No. | Water | HCl | NH₄ | alcohol | Acetone | Toluene | spirit | oil |
| 1 | − | − | − | − | − | −/+ | − | −/+ |
| 2 | − | − | − | − | − | − | − | −/+ |
| 3 | − | − | − | − | − | + | − | −/+ |
| 4 | − | − | − | − | −/+ | − | − | −/+ |
| Order* | 1, 3, 2, 4 | 1, 3, 2, 4 | 2, 3, 4, 1 | 4, 1, 3, 2 | 1, 2, 3, 4 | 4, 2, 1, 3 | 1, 2, 3, 4 | 1, 2, 3, 4 |

+ = Medium blue color observed.
−/+ = Light blue color observed.
− = Very clear or very light blue color observed.
*The order indicates the sample numbers from the clearest sample to the bluest sample.

Because methylene blue is a very dark blue dye, it was clear from the above results that the organoclay was tightly holding onto the colored methylene blue particles. This is because after the colored organoclay was centrifuged out of the given solvents and the test tubes of each of the remaining liquid solvent were visually inspected, none of the liquids were dark blue in color. Thus, in all of the cases described above (regardless of the order of the addition of the ingredients and regardless of the liquid used to disperse each methylene blue/organoclay composition), the methylene blue dye had successfully colored the organoclay and was not “bleeding” or being pulled away from the clay by any of the solvents, even though the organoclay/methylene blue compositions were highly dispersible in each of the solvents.

Thus, in general the results shown in Table 6 and in the above Example reveal that the methylene blue was strongly affixed to the surface of the organoclay and was not extracted to any appreciable extent by any of the 8 different dispersal fluids used. Also, there were no appreciable differences in performance as the order of the addition of the ingredients was varied for Samples 1-4. For example, even when a 10% excess of quat was used for Sample 4, the cationic methylene blue dye was still tightly bound to the organoclay.

We claim:

1. A composition comprising:
i) a cationic molecule of interest,
ii) a cationic organic molecule; and
iii) a high surface area substrate;
wherein the cationic molecule of interest and the organic cationic compound are chemically bound to the high surface area substrate.

2. The composition of claim 1, wherein said substrate is a silicate.

3. The composition of claim 2, wherein said silicate is zeolite.

4. The composition of claim 1, wherein said substrate is a clay.

5. The composition of claim 1, wherein said organic cationic compound comprises a cation selected from the group consisting of quaternary ammonium, quaternary phoshponium, and ternary sulfonium.

6. The composition of claim 1, wherein said organic cationic compound comprises a linear, saturated alkyl moiety having 12 to 22 carbon atoms and one or more moieties selected from the group consisting of a linear, saturated alkyl moiety having 12 to 22 carbon atoms; a branched, saturated alkyl moiety having 12 to 22 carbon atoms; a linear, unsaturated alkyl moiety having 12 to 22 carbon atoms; a branched, unsaturated alkyl moiety having 12 to 22 carbon atoms; a benzyl moiety including fused ring moieties having linear or branched 1 to 22 carbon atoms in the alkyl portion of the structure; a substituted benzyl moiety including fused ring moieties having linear or branched 1 to 22 carbon atoms in the alkyl portion of the structure; a phenyl moiety and substituted phenyl including fused ring aromatic substituents; a substituted phenyl moiety including fused ring aromatic substituents; a beta, gamma-unsaturated moiety having six or less carbon atoms or hydroxyalkyl groups having two to six carbon atoms; and hydrogen.

7. The composition of claim 1, wherein said organic cationic compound comprises a branched, saturated alkyl moiety having 12 to 22 carbon atoms and one or more moieties selected from the group consisting of a linear, saturated alkyl moiety having 12 to 22 carbon atoms; a branched, saturated alkyl moiety having 12 to 22 carbon atoms; a linear, unsaturated alkyl moiety having 12 to 22 carbon atoms; a branched, unsaturated alkyl moiety having 12 to 22 carbon atoms; a benzyl moiety including fused ring moieties having linear or branched 1 to 22 carbon atoms in the alkyl portion of the structure; a substituted benzyl moiety including fused ring moieties having linear or branched 1 to 22 carbon atoms in the alkyl portion of the structure; a phenyl moiety and substituted phenyl including fused ring aromatic substituents; a substituted phenyl moiety including fused ring aromatic substituents; a beta, gamma-unsaturated moiety having six or less carbon atoms or hydroxyalkyl groups having two to six carbon atoms; and hydrogen.

8. The composition of claim 1, wherein said organic cationic compound comprises a linear, unsaturated alkyl moiety having 12 to 22 carbon atoms and one or more moieties selected from the group consisting of a linear, saturated alkyl moiety having 12 to 22 carbon atoms; a branched, saturated alkyl moiety having 12 to 22 carbon atoms; a linear, unsaturated alkyl moiety having 12 to 22 carbon atoms; a branched, unsaturated alkyl moiety having 12 to 22 carbon atoms; a benzyl moiety including fused ring moieties having linear or branched 1 to 22 carbon atoms in the alkyl portion of the structure; a substituted benzyl moiety including fused ring moieties having linear or branched 1 to 22 carbon atoms in the alkyl portion of the structure; a phenyl moiety and substituted phenyl including fused ring aromatic substituents; a substituted phenyl moiety including fused ring aromatic substituents; a beta, gamma-unsaturated moiety having six or less carbon atoms or hydroxyalkyl groups having two to six carbon atoms; and hydrogen.

9. The composition of claim 1, wherein said organic cationic compound comprises a branched, unsaturated alkyl moiety having 12 to 22 carbon atoms and one or more moieties selected from the group consisting of a linear, saturated alkyl moiety having 12 to 22 carbon atoms; a branched, saturated alkyl moiety having 12 to 22 carbon atoms; a linear, unsaturated alkyl moiety having 12 to 22 carbon atoms; a branched, unsaturated alkyl moiety having 12 to 22 carbon atoms; a benzyl moiety including fused ring moieties having linear or branched 1 to 22 carbon atoms in the alkyl portion of the structure; a substituted benzyl moiety including fused ring moieties having linear or branched 1 to 22 carbon atoms in the alkyl portion of the structure; a phenyl moiety and substituted phenyl including fused ring aromatic substituents; a substituted phenyl moiety including fused ring aromatic substituents; a β, γ-unsaturated moiety having six or less carbon atoms or hydroxyalkyl groups having two to six carbon atoms; and hydrogen.

10. The composition of claim 4, wherein said clay is selected from the group consisting of a bentonite, a montmorillonite, a beidellite, a hectorite, a saponite, a stevensite, and mixtures thereof.

11. The composition of claim 1, wherein said cationic molecule is a component of a dye.

12. The composition of claim 1, wherein said cationic molecule is a component of a pigment.

13. The composition of claim 1, wherein said cationic molecule is a component of a catalyst.

14. The composition of claim 1, wherein said cationic molecule is a component of a redox agent.

15. The composition of claim 1, wherein said cationic molecule is a component of a medicinal substance.

16. The composition of claim 11, wherein said dye is selected from the group consisting of methylene blue, basic Yellow 57, Jarocol Staw Yellow, Basic Green 4, Basic Red 104, methyl green, pyocyanine, phenosafranin and celestine blue.

17. The composition of claim 15, wherein said medicinal substance is selected from the group consisting of zinc ricinoleicite, ricinoleic acid, calcium ethylbutanoate, aluminum nicotinate.

18. A method of increasing the surface area of an cationic molecule of interest in an application system, comprising bonding the cationic molecule of interest and the cationic organic compound to a high surface area substrate wherein said substrate is capable of cation exchange because of mobile cations located at its surface; thereby forming a composition comprising a complex between the cationic molecule of interest, the cationic organic compound and the high surface substrate complex wherein the cationic molecule of interest incorporated onto the high surface area substrate displays an enhanced surface area in said application system than the cationic molecule of interest would display alone.

19. The product produced by the method of claim 18 wherein the cationic molecule of interest has an enhanced surface area.

20. The method of claim 18 wherein the enhanced surface area of the cationic molecule of interest confers a desired physical, chemical, biological or therapeutic benefit to an application system.

21. The method of claim 20, wherein said physical, chemical, biological or therapeutic activity is selected from the group consisting of optical activity, insolubility, catalytic activity, oxidative activity, reductive activity, anti-cholinergic activity, anti-spasmodic activity, anti-microbial activity, anti-fungal activity, muscle-relaxant activity, disinfectant activity, anti-bacterial activity, leachability and dispersibility.

22. The method of claim 18 for producing a dry powder pigment.

23. A dry powder pigment produced by the method of claim 22.

24. The method of claim 18 for producing a dry powder colorant.

25. A dry powder colorant produced by the method of claim 24.

26. The method of claim 18 for reducing the leachibility of a cationic molecule of interest in an application system.

27. A product produced by the method of claim 26 wherein the cationic molecule of interest has reduced leachibility.

28. A method of tinting a plastic comprising incorporating the dry powder pigment of claim 23 into said plastic.

29. A method of tinting a polymer comprising incorporating the dry powder pigment of claim 23 into said polymer.

30. A method of tinting a resin comprising incorporating the dry powder pigment of claim 23 into said said resin.

31. A tinted plastic produced by the method of claim 28.

32. A tinted polymer produced by the method of claim 29.

33. A tinted resin produced by the method of claim 30.

34. A method of tinting a plastic comprising incorporating the dry powder colorant of claim 25 into said plastic.

35. A method of tinting a polymer comprising incorporating the dry powder colorant of claim 25 into said polymer.

36. A method of tinting a resin comprising incorporating the dry powder colorant of claim 25 into said resin.

37. A tinted plastic produced by the method of claim 34.

38. A tinted polymer produced by the method of claim 35.

39. A tinted resin produced by the method of claim 36.
